(19) United States
(12) Patent Application Publication
(10) Pub. No.: US 2004/0028550 A1
(43) Pub. Date: Feb. 12, 2004

(54) Air purification with ozone

(76) Inventors: Robert Malcolm Thomas, Alvin, TX (US); Karl Frederick Thomas, Alvin, TX (US)

Correspondence Address: NICK A NICHOLS, P O BOX 16399, SUGARLAND, TX, 774966399

(21) Appl. No.: 10/410,869

(22) Filed: Apr. 10, 2003

Related U.S. Application Data
(60) Provisional application No. 60/371,464, filed on Apr. 10, 2002.

(51) Int. Cl. A61L 9/015

(52) U.S. Cl. 422/4

(57) ABSTRACT

In a process for generating an effective concentration of ozone for deodorization and destruction of contaminants. The contaminants are covered by an enclosure which is about the perimeter thereof. A conduit connected to an ozone generator is inserted into the enclosure and the distal end of the conduit is positioned at about the center of the area of contamination. Ozone is directed into the contaminant enclosure for a sufficient period of time to destroy the contaminant and deodorize the enclosed area.

### CROSS-REFERENCE TO RELATED APPLICATION

This application claims the benefit of U.S. Provisional Application Serial No. 60/371,464, filed Apr. 10, 2002, which application is incorporated herein by reference.

### BACKGROUND OF THE DISCLOSURE

The present invention relates to a process for air purification. More particularly, the invention relates to a process for decontamination and/or remediation of mold and mildew and elimination of odors and other contaminants using ozone.

Many attempts have been made to reduce offensive ordors and infectious agents in enclosed spaces. The methods include disinfecting agents, odor masking agents, activated chemical filters and oxidants. Ozone is a well known oxidant that destroys bacteria, viruses and other microorganisms on contact. Ozone, or O3, is an allotropic form of oxygen produced from air or other gasses containing oxygen by passage of a stream of such gasses through a high voltage electrical discharge. Contaminants, such as bacteria, which come in contact with ozone are ruptured in a process known as cell lysing. Destruction of the contaminant is almost immediate. This and other benefits of ozone are well known. However, unlike many other disinfecting agents, ozone is used as it is generated, breaks down rapidly and does not leave chemical residues.

The effectiveness of mold and mildew remediation using ozone, however, has been debated for some time. Thus, a need remains for a process or protocol that utilizes ozone which effectively remediates mold and mildew and other contaminants and odors.

It is therefore an object of the present invention to provide a process for remediation of odors and contaminants using ozone.

### SUMMARY OF THE INVENTION

In accordance with the present invention, a process for generating an effective concentration of ozone for deodorization and destruction of contaminants is provided. The process includes forming a deodorization or decontamination enclosure about an object or contaminated area and sealing the perimeter thereof. A conduit or tube having one end connected to an ozone generator is inserted under the covering and the distal end thereof is positioned at or about the center of the enclosed area. Ozone is directed into the enclosure for a sufficient period of time to deodorize the object and/or destroy the contaminant.

### DETAILED DESCRIPTION OF A PREFERRED EMBODIMENT

In a preferred embodiment of the invention ozone is utilized for the remediation of contaminants, such as mold and mildew fungi. There are many kinds of molds and mildews, some of which, such as stachybotrys, can be deadly. Many methods of controlling fungi, such as various chemical sprays, have been used with various levels of success. Chemical sprays, however, may result in exposing occupants of the treated area to chemical fumes which may be as toxic or allergyinducing as those produced by molds, mildews, odors and other contaminants.

In the process of the present invention, ozone is used to deodorize and decontaminate an affected area. Ozone not only oxidizes the body of the fungi organism, but the fungal roots and spores as well. Ozone has a half-life of about thirty-six minutes in air and then begins to revert to natural oxygen. No film or fumes are left behind to cause respiratory problems in individuals who may be sensitive to chemicals.

In a preferred embodiment for the remediation of mold and mildew according the process of the present invention, an initial viable and non-viable investigation/test on each mold and mildew area is made to determine the level of contamination. The affected area of mold and mildew, such as a wall, floor or other surface, is then covered with a non-permeable material, such as plastic, and the perimeter about the affected area is sealed to completely enclose the mold and mildew. Care should be taken to minimize disturbing the mold and mildew so that it is contained under the plastic cover and not permitted to become air-borne. The plastic cover placed over the area of mold and mildew is preferably supported above the affected area by support or brace members, a temporary perimeter support structure or the like. In a preferred embodiment, the covering material is supported by 2×4 support beams located about the perimeter of the mold and mildew area forming a tent-like enclosure over the affected area. The covering material is secured about the affected area with tape or the like, such as duct tape used in HVAC work, and completely encloses the mold and mildew area. While use of a support structure for the enclosure covering is preferred, it is understood that a support structure may be omitted under some circumstances, particularly where placement of the enclosure covering over the affected area will not significantly disturb the mold and mildew causing it to become air borne.

The entire perimeter of the covering material is sealed with the tape or the like about the mold and mildew area except for a small area providing an opening for insertion of a conduit, such as a tube or the like, under the covering material for injecting ozone into the mold and mildew enclosure. The distal end of the ozone injection conduit is positioned at or about the center of the mold and mildew area with its end pointed upward toward the underside of the plastic covering over the mold and mildew area.

The ozone injection conduit is connected at its opposite end to an ozone generator. Preferably, the ozone generator is of the type manufactured by Marhoc, Inc. which utilizes ambient air that is dried prior to input into the ozone reactor for generating ozone, described in greater detail in U.S. Pat. No. 6,120,739 issued Sept. 19, 2000. Dried air input into the ozone reactor increases the ozone output and decreases nitrogen byproducts typically generated when air containing moisture is input into the ozone reactor to generate ozone. Low nitrogen generation is important because moist nitrogen containing environments are ideal for stachybotrys fungi growth.

The ozone output from the ozone generator is directed through the injection conduit into the sealed enclosure over the mold and mildew area. The distal end of the injection conduit is pointed upward so that ozone exiting therefrom is reflected off of the inside of the covering plastic sheet toward the mold and mildew, rather than being injected directly on the mold and mildew, thereby creating an indirect treatment effect. The ozone accumulating within the sealed enclosure actually “smothers” the mold and mildew rather than blowing it around inside the containing enclosure. Ozone is injected into the mold and mildew enclosure for a predetermined period of time sufficient to destroy the mold and mildew.

At the end of the run cycle of the ozone generator, the same investigations/tests that were initially conducted of the mold and mildew area prior to treatment with ozone are repeated. If the test results show that the mold and mildew is destroyed, the mold and mildew area is vacuumed with a HEPA vacuum or the like. An air blower is then used to blow air directly on the affected area, which area is treated with dry air for a period of time, preferably for at least two hours. If the test results are not conclusive as to the effectiveness of the mold and mildew remediation and contaminant level are not within acceptable limits, the process is repeated.

The process of the present invention is suitable for use in any space which may be reached with the ozone injection conduit connected to the ozone generator. For example, the process of the invention may be utilized for remediation of mold and mildew in a cellar, attic, closet, inside walls of a structure, underneath and/or outside of a facility, or any other remote location which is enclosed or may be enclosed by a covering material. Likewise, the process of the present invention may also be used after a fire for smoke and odor remediation of a structure and/or furniture. An ozone enclosure may easily be formed over furniture for on-the-spot deodorization by draping a plastic cover over the furniture and sealing the perimeter thereof in the manner described above. A small hole, about the size of the tip of a ball point pen, may be formed in the plastic covering for pressure release to prevent the plastic cover from bursting. The deodorization process then proceeds as described above.

While a preferred embodiment of the invention has been shown and described, other and further embodiments of the invention may be devised without departing from the basic scope thereof, and the scope thereof is determined by the claims which follow.

1. An air purification process, comprising the steps of:
a) locating the presence of a contaminant;
b) covering the contaminant to form an enclosure over the contaminant having a perimeter circumscribing the contaminant;
c) sealing the perimeter of the enclosure;
d) introducing ozone into the enclosure for a predetermined period;
e) determining whether the level of contaminant within the enclosure after introdicution of ozone ceases is within acceptable limits;
f) removing the enclosure about the contaminant; and
g) disposing of any contaminant remains.

2. The process of claim 1 wherein the contaminant is mold.

3. The process of claim 1 wherein the contaminant is mildew.

4. The process of claim 1, including the step of conducting viable and non-viable investigations for the contaminant prior to introduction of ozone.

5. The process of claim 1, including the step of introducing ozone through a conduit inserted into the enclosure and injecting ozone through the conduit directed upward against the underneath surface of the enclosure.

6. The process of claim 1, including the step of utilizing dried input air into an ozone generator for creating the ozone introduced into the enclosure covering the contaminant.
